(19) United States
(12) Patent Application Publication
(10) Pub. No.: US 2005/0003521 A1
(43) Pub. Date: Jan. 6, 2005

(54) Addressable microarray device, methods of making, and uses thereof

(76) Inventors: David O'Connor, North Bend, WA (US); Barton Norton, Redmond, WA (US); Gerald Klein, Edmonds, WA (US)

Correspondence Address: GRAY CARY WARE & FREIDENRICH LLP, 2000 UNIVERSITY AVENUE, E. PALO ALTO, CA, 94303-2248, US

(21) Appl. No.: 10/798,639

(22) Filed: Mar. 10, 2004

Related U.S. Application Data
(60) Provisional application No. 60/453,932, filed on Mar. 11, 2003.

(51) Int. Cl. C12Q 1/68; C12M 1/34

(52) U.S. Cl. 435/287.2; 435/6

(57) ABSTRACT

The present invention relates to devices and methods for performing an array of chemical reactions. The device includes a substrate having an array of microwells. Each microwell within the array includes a porous region defined in the first side and extending partially through the substrate. The porous region is formed by the selective removal of a substrate constituent, such that the porous region is defined by a continuous portion of the substrate. A wide range of functional groups, sample molecules, and chemical moieties that can be easily introduced into the described microwells and immobilized therein, particularly onto the porous region of the substrate, therefore the devices of the present invention are useful as supports for the synthesis of compounds, such as biomolecules, and for a range of methods involving chemical reactions and assays.

### CROSS REFERENCE TO RELATED APPLICATION

This application claims priority under 35 U.S.C. § 119(e)(1) to U.S. Provisional Application Ser. No. 60/453,932, filed Mar. 11, 2003, herein incorporated by reference in its entirety.

### BACKGROUND OF THE INVENTION

1. Field of the Invention

The invention relates generally to microarrays for synthesizing and analyzing molecules, and more specifically to methods and devices having an array of microwells suitable for performing a plurality of chemical reactions, assays, and synthesis reaction.

2. Background Information

Chemical analysis, detection and synthesis of biomolecules has become very important in research and in many industries, and the analysis of biological molecules such as nucleic acids and proteins forms the basis of various assays. The procedures utilized often involve large numbers of repetitive steps which consume large amounts of time and resources. (see, e.g., Sambrook, J., et al., Molecular Cloning: A Laboratory Manual. Cold Spring Harbor Laboratory Press, Cold Spring Harbor, N.Y. (2nd ed. 1989)). Simpler and quicker analysis of molecules has been provided by the development of arrays of test sites formed on a planar substrate. Each of the test sites includes probes which bind with samples applied to the device. Such probes may be oligonucleotides, proteins, antibodies, or cell-binding molecules and the choice of probes is theoretically limited only by the possibilities of specific binding to or reaction with sample. The binding of a sample to a probe is detected, and the probe identified, thereby identifying the sample. Technology has primarily developed around the use of these two-dimensional, planar arrays, especially in the area of arrays of oligonucleotides, which have become small and dense enough to be termed microarrays.

The ability to manufacture microarrays in an efficient and cost-effective manner is of considerable interest to researchers worldwide and of significant commercial value. The importance of the microarray technology to the biotechnology industry and to the entire health care sector cannot be overstated. A microarray is capable of dramatically boosting the efficiency of traditional biochemical experiments. Tests that would have taken years can now be completed in hours or even minutes. The applications of this technology affect more than the healthcare sector including gene profiling, disease diagnostics, drug discovery, forensics, agronomics, biowarfare and even biocomputers.

Various types of microarray manufacturing devices and technologies have been described. However, there is a continuing need for microarrays having added functionality and capable of being manufactured in cost-effective manner. In particular, there is a need for improved devices and methods involving microarrays suitable for microvolume chemical reactions.

### SUMMARY OF THE INVENTION

One aspect of the present invention relates to a device for performing an array of chemical reactions. The device includes a substrate having a first side, a second side, and an array of microwells. Each microwell within the array includes a porous region defined in the first side and extending partially through the substrate. The porous region is formed by the selective removal of a substrate constituent, such that the porous region is defined by a continuous portion of the substrate. Further, each microwell is capable of holding a sample such that a liquid sample from one microwell does not intermix with a liquid sample from another microwell. Because of the wide range of functional groups, sample molecules, and chemical moieties that can be easily introduced into the described microwells and immobilized therein, particularly onto the porous region of the substrate, the devices of the present invention are useful as supports for the synthesis of compounds, such as biomolecules, and for a range of methods involving chemical reactions and assays.

A microwell of the substrate can further include a cavity located on the second side of the substrate, where the cavity extends partially through the substrate as to intersect with the porous region on the first side of the substrate. Thus, in one embodiment, a microwell includes a porous region and a cavity that are aligned such that the microwell forms a continuous channel extending through the substrate. The open channel formed by the aligned porous region and cavity are capable of forming an ion bridge between the two sides of the substrate.

Other embodiments of the invention include methods of producing a device for performing an array of chemical reactions. These methods include providing a substrate having a first side, a second side, and forming an array of microwells in the substrate. In one embodiment, the microwells are formed by selectively leaching defined areas on the first side of the substrate, thereby forming a plurality of porous regions that are a continuous portion of the substrate and extend partially through the substrate. The method further includes selectively etching defined areas of the second side of the substrate, thereby forming a plurality of cavities. Each of the defined cavities on the second side extend partially through the substrate to intersect with a porous region on the first side. Thus each microwell is formed from a porous region aligned with a cavity, such that the porous region and the cavity form an open channel extending through the substrate.

Other methods of the invention include methods that are carried out in a device of the invention, or in a substrate having an array of microwells. In one embodiment, such methods include simultaneously conducting a plurality of chemical reactions. These methods include providing a substrate having an array of microwells, each microwell having a porous region formed in a first side of the substrate and capable of binding a sample molecule, where the porous region is a continuous portion of the substrate, extends partially through the substrate, and is formed by selectively removing at least one constituent of the substrate. Each microwell further includes a cavity located at a side of the substrate opposite the first side, with the cavity extending partially through the substrate to intersect the porous region. The methods further include introducing, under suitable reaction conditions, a plurality of test samples into the a plurality of microwells of the device, wherein the test samples contain sample molecules as well as necessary reaction components.

One method includes detecting the presence or amount of an analyte in an array of test samples. The method includes contacting a test sample, under suitable binding conditions, with in the array of samples with a microwell defined in a substrate of the invention. Such a substrate includes an array of microwells, where each microwell includes a porous region and a cavity. The porous region of each microwell is formed in a first side of the substrate by selectively removing at least one substrate constituent. Further, the porous region is a continuous portion of the substrate, and extends partially through the substrate. The cavity of a microwell is located at a side of the substrate opposite the first side, and each cavity extends partially through the substrate to intersect the porous region. Each microwell further includes a probe immobilized to the porous region. The method also includes forming a complex between the probe and the analyte, and detecting the probe-analyte complex, thereby detecting the presence or amount of an analyte in a test sample.

In another aspect of the invention, a method of assembling a compound is provided. The method includes providing a substrate having an array of microwells, each microwell having a porous region formed in a first side of the substrate and capable of binding a component of a compound, where the porous region is a continuous portion of the substrate, extends partially through the substrate, and is formed by selectively removing at least one constituent of the substrate. Each microwell further includes a cavity located at a side of the substrate opposite the first side, with the cavity extending partially through the substrate to intersect the porous region. The method further includes adding a first component of the compound into a plurality of microwells of the substrate, such that the added first component is immobilized to porous regions of the microwells; adding a second component of the compound to the microwells; and reacting, within each microwell of the plurality of microwells, the first component and the second component to form a product, thereby assembling a plurality of compounds.

In another aspect of the invention, a kit comprising a device for performing chemical reactions is provided. The device of the kit includes a substrate having an array of microwells. Each microwell of the array includes a porous region and a cavity. The porous region is formed in a first side of the substrate and capable of binding sample molecules, wherein the porous region is a continuous portion of the substrate, extends partially through the substrate, and is formed by selectively removing at least one constituent of the substrate. The cavity of each microwell is located at a side of the substrate opposite the first side and extending partially through the substrate to intersect the porous region. The kit further includes a reaction component packaged in a suitable container. The reaction component can be a reagent for performing various reactions, including ligation reactions, primer extension reactions, nucleotide sequencing reactions, restriction endonuclease digestions, oligonucleotide syntheses, hybridization reactions and biomolecular interactions.

### BRIEF DESCRIPTION OF THE DRAWINGS

FIG. 1 illustrates a cross-sectional view of a substrate having an array of microwells.

FIGS. 2a and 2b illustrate a device according to an embodiment of the present invention.

FIGS. 3a and 3b illustrate the process of fabricating a device according to an embodiment of the present invention.

### DETAILED DESCRIPTION OF THE INVENTION

One aspect of the present invention relates to a device for performing an array of chemical reactions. The device includes a substrate having a first side, a second side, and an array of microwells. Each microwell within the array includes a porous region defined in the first side and extending partially through the substrate. The porous region is formed by the selective removal of a substrate constituent, such that the porous region is defined by a continuous portion of the substrate. Further, each microwell is capable of holding a sample such that a liquid sample from one microwell does not intermix with a liquid sample from another microwell.

A microwell of the substrate can further include a cavity located on the second side of the substrate, where the cavity extends partially through the substrate as to intersect with the porous region on the first side of the substrate. Thus, in one embodiment, a microwell includes a porous region and a cavity that are aligned such that the microwell forms a continuous channel extending through the substrate. The open channel formed by the aligned porous region and cavity are capable of forming an ion bridge between the two sides of the substrate.

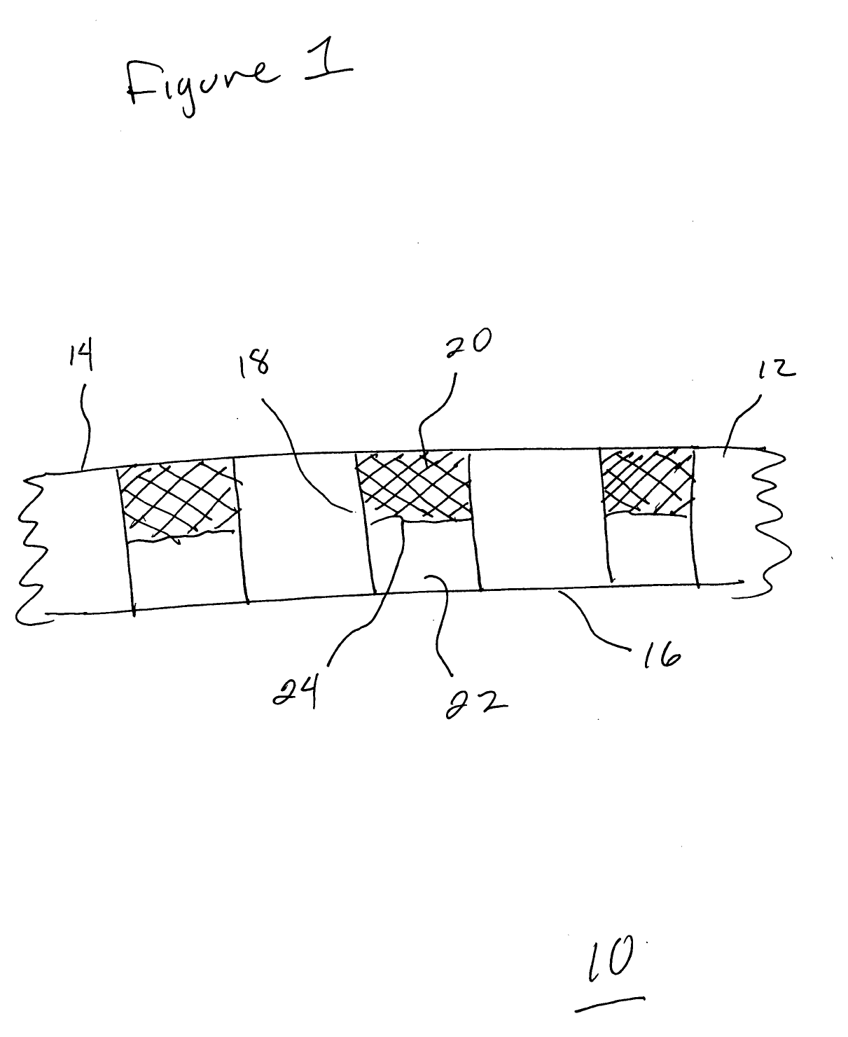

With reference to FIG. 1, a device 10 comprises a substrate 12 with a first side 14 and a second side 16, and an array of microwells. Each microwell 18, includes a porous region 20 and a cavity 22. A well bottom 24 is an area within a microwell where the porous region contacts the cavity.

The term “substrate” or “microfabricated substrate”, as used herein, refers to a substrate having an array of microwells defined therein. Particular substrates that are useful in practicing the present invention can be made of practically any physicochemically stable material capable of forming a porous region thereon by the selective removal of certain substrate constituents. Substrates can include optically opaque substrates, optically transparent substrates, insulating substrates, conducting substrates, semiconducting substrates, magnetic substrates and combinations thereof.

Useful substrates are not limited to a particular size or range of sizes. The choice of an appropriate substrate size for a given application will be apparent to those of skill in the art. The substrate can be any shape including, for example, round, oval, wafer-like, square, rectangular, and the like. In certain embodiments, substrate of the invention is substantially square, meaning the length and width are approximately equal. In another embodiment, the substrate is elongated or substantially rectangular, or the length is greater then the width. In other preferred embodiments, the substrate is substantially rectangular having a length of about 75 mm and a width of about 25 mm.

Exemplary substrate materials include, but are not limited to, inorganic crystals, inorganic glasses, inorganic oxides, metals, organic polymers and combinations thereof. Inorganic crystals and inorganic glasses that are appropriate for substrate materials include, for example, LiF, NaF, NaCl, KBr, KI, CaF₂, MgF₂, HgF₂, BN, AsS₃, ZnS, Si₃N₄ and the like. The crystals and glasses can be prepared by art standard techniques. See, for example, Goodman, Crystal Growth Theory and Techniques, Plenum Press, New York, 1974. Alternatively, the crystals and glasses can be purchased commercially (e.g., Fisher Scientific, Duke Scientific Corporation, Palo Alto, Calif.). Inorganic oxides can also form a substrate of the device of the present invention. Inorganic oxides of use in the present invention include, for example, Cs₂O, Mg(OH)₂, TiO₂, ZrO₂, CeO₂, Y₂O₃, Cr₂O₃, Fe₂O₃, NiO, ZnO, Al₂O₃, SiO₂ (glass), silica, borosilcate glass, quartz, In₂ O₃, SnO₂, PbO₂ and the like. A substrate can consist of a single inorganic oxide or a composite of more than one inorganic oxide. For example, a composite of inorganic oxides can have a layered structure (i.e., a second oxide deposited on a first oxide) or two or more oxides can be arranged in a contiguous non-layered structure. In addition, one or more oxides can be admixed as particles of various sizes and can optionally be deposited on a support such as a glass or metal sheet. In one embodiment, an inorganic oxide can be admixed with a metallic or semi-metallic element or constituent (e.g., boron). Further, a layer of one or more inorganic oxides can be intercalated between two other substrate layers (e.g., metal-oxide-metal, metal-oxide-crystal). Appropriate inorganic oxide particles can be prepared or, alternatively, they can be purchased from commercial sources (e.g., Duke Scientific Corporation, Palo Alto, Calif.).

In one embodiment, the substrate is a silicon oxide glass, and more particularly a borosilicate glass. Even more particularly, the substrate of the invention can include a borosilicate microscope slide, such as is generally commercially available (e.g., Precision Glass & Optics, Santa Ana, Calif.).

Microwells can be formed in any arrangement within a substrate that is suitable for the experimental purpose of the device. For example, microwells are arranged in rows and columns on a rectangular substrate. In one particular illustrative example, a microfabricated substrate includes 1536 microwells arranged in an array of 64 columns by 24 rows. The number and arrangement of microwells on a substrate can vary, and is designed with the particular experimental use in mind.

The size of the microwell is commensurate with the reaction volume and can be varied by varying the width (or diameter) of the microwell and/or the thickness of the substrate (which effectively varies the height or depth of the microwell where the microwell extends through the substrate). Thus, the volume of a sample which can be contained in a microwell is a function of the height of the microwell and the width of the microwell. However, a microwell can be loaded such that the liquid extends beyond the physical boundaries of the microwell; in some cases this will be facilitated if the surface of the substrate surrounding the openings of the microwells comprises a sample containment layer, such as a hydrophobic material; in other cases, it will be accomplished by surface tension. In this fashion, a volume of liquid which is greater than the volume of the microwell can be accommodated by a sample chamber. Conversely, a microwell can be loaded with a volume of liquid that is less than the volume of the microwell. Accordingly, sample volumes of less than about 100 microliters, for example, less than 100 nanoliters, less than about 50 nanoliters, less than about 100 picoliters, less than about 75 picoliters, less than about 25 picoliters can be reliably achieved. In one embodiment, sample volumes as low as about 1 picoliter can be used. Thus, sample volume contemplated range includes from about 100 microliters to about 1 picoliter, and typically about 100 nanoliters to about 25 picoliters, or about 100 picoliters to about 25 picoliters.

The fabrication of the array of microwells on a substrate can be accomplished according to various techniques. For example, array formation can be accomplished by using masking and fabrication techniques such as photolithography (Kleinfield et al., J. Neurosci. 8:4098-120 (1998)), photoetching, microlithograph, chemical etching and microcontact printing (Kumar et al., Langmuir 10:1498-511 (1994)). Other techniques for forming a microwell array on a substrate will be readily apparent to those of skill in the art.

Thus the term “mask” or “masking”, as used herein, refers to a means of selectively forming a pattern covering an area or surface of a substrate of the invention, where the mask pattern, when coupled with microwell fabrication methods (e.g., leaching, etching), allows fabrication of microwells on the substrate as specified by the mask. The mask is typically resistant to agents or chemicals used in leaching or etching, such that application of a mask followed by leaching and/or etching allows selective leaching and/or etching at the exposed regions of the substrate, or regions not having a mask. For example a mask pattern can be printed directly onto the substrate or, alternatively, a “lift off” technique can be utilized. In the lift off technique, a patterned mask is laid onto the substrate, an organic layer is laid down in those areas not covered by the mask and the mask is subsequently removed. Masks appropriate for use with the substrates of the present invention are known to those of skill in the art. See, for example, Keinfield et al., J. Neurosci. 8:4098-120 (1998).

In one embodiment, fabrication methods are used to produce a substrate having a plurality of adjacent microwells, wherein each of these features is isolated from the other microwells and the wells do not fluidically communicate. Thus, a sample or substance, including those contained in a liquid sample, placed in a particular microwell remains substantially confined to that well. In an embodiment where a microwell comprises a porous region and a cavity aligned to form a channel extending through the substrate, positioning of additional components in the cavity, such as an electrode, can prevent a liquid sample from flowing out of the microwell through the cavity. In another embodiment, the patterning allows the creation of channels through the device whereby an analyte or sample can enter and/or exit the device.

Therefore, present invention additionally includes methods of producing a device for performing an array of chemical reactions. These methods include providing a substrate having a first side, a second side, and forming an array of microwells in the substrate by selectively leaching the first side, and selectively etching the second side. The microwells may be formed by selectively leaching defined areas on the first side of the substrate, thereby forming a plurality of porous regions that are a continuous portion of the substrate and extend partially through the substrate.

An array of microwells having a porous region may be formed on a side of substrate through chemical dissolution or leaching. The term “porous” as in a “porous region” as used herein refers to a region of a microwell defined in a microfabricated substrate of the invention, having a porosity (void percentage) in the range of about 1% to about 99%, preferably about 5% to about 99%, more preferably in the range of about 15% to about 95%. Furthermore, pore size generally at least 1 nanometer (nm), and ranges from about 1 nm to about 100 nm. More particularly, pore size can be at least 2.5 nm, and typically between 2.5 nm and 60 nm, typically about 5 nm, 10 nm, 20 nm, 30 nm, 40 nm, or 50 nm. A porous region of a microwell is formed by selectively removing a constituent of the substrate.

The terms “leaching”, “chemical leaching”, and “chemical dissolution”, as used herein, refer to the processes of selectively removing at least one constituent of a substrate. More specifically, leaching or chemical dissolution includes removing, or dissolving out, a substrate constituent, such as soluble constituents, by subjecting the substrate to the action of a gas, fluid or liquid composition. By exposing a substrate to a suitable leachant, or dissolving chemical, certain substrate constituents are removed while others remain in place as a continuous portion of the substrate. The substrate constituent desired to be removed will depend on the composition of the substrate and will be apparent to one skilled in the art. The substrate constituent to be selectively removed, for example, can include, without limitation, a metallic or semi-metallic element or constituent. In one embodiment, where the substrate is borosilicate glass, the selectively removed constituent includes boron ions. Leaching is a diffusion-limited process where the depth of leaching of a substrate can be controlled by adjusting the time, temperature and concentration parameters on the leachant. Various methods of leaching a substrate, such as those used in forming controlled-pore glasses (CPG), will be readily apparent to one of skill in the art. See, for example, Vashneya, Fundamentals of Inorganic Glasses, Academic Press, 1993, which is hereby incorporated by reference.

In one embodiment, the substrate may comprise an inorganic glass, including a composite glass such as borosilicate glass, that is heat-treated prior to leaching. For example, various inorganic glasses suitable as substrates of the invention can be additionally processed prior to the step of leaching by heating the substrate to a temperature above the substrate annealing temperature, but below the softening temperature, such that constituent ions in the substrate migrate and coalesce together, forming coalesced constituents that can be further subjected to leaching. Leaching may then be accomplished, for example, by contacting the heat-treated substrate with a leachant, such as an acid (e.g., H₂SO₄) or a base, such that the coalesced constituents of the heat-treated substrate are dissolved, leaving a porous structure.

Additionally, the substrate may be selectively leached such that leaching occurs only at predetermined areas of the substrate. Selective leaching may be accomplished, for example, by masking, as discussed above. Therefore, in one embodiment, producing a substrate having an array of microwells includes applying a mask to a surface of a substrate, where the mask partially covers the substrate surface, and contacting the surface with a leachant, wherein the leachant forms porous microwells at areas of the surface not covered by the mask, thereby selectively leaching a surface of a substrate.

A method can further include selectively removing predetermined and defined areas of a substrate, thereby forming a plurality of cavities. Each of the cavities formed on the substrate extend partially through the substrate to intersect with a porous region on the opposite side of the substrate. Thus each microwell is formed from a porous region aligned with a cavity, such that the porous region and the cavity form an open channel extending through the substrate.

Selective removal of substrate material as to form a plurality of cavities can be accomplished by various techniques known in the art, including fabrication techniques discussed above. The substrate may be selectively etched, for example, by applying a mask to a side of the substrate and contacting the masked substrate with an etchant. For example, producing a cavity can include applying a mask to a surface of a substrate, where the mask partially covers the substrate surface, and contacting the surface with a etchant, wherein the etchant removes areas of the surface not covered by the mask, thereby selectively etching a surface of a substrate. In one embodiment, cavities are formed by etching with a suitable chemical etchant, including, for example, a ammonium bifluoride solution or hydrofluoric acid solution. Other suitable etchants will be readily apparent to one skilled in the art.

A method of the invention may optionally include an addition step of increasing the size of pores in the porous regions of the microwells. Increasing pore size may be desired in some instances, for example, where the pore size produced by leaching alone is too small for the molecules, such as larger biomolecules, being immobilized in the microwell. Increasing pore size may be accomplished by a variety of techniques and can include, for example, contacting the porous region of a microwell with an etchant. Where a chemical etchant is used, the increased size of the pores due to further etching of the porous region is dependent on factors such as duration of exposure, temperature, and concentration of the etchant.

The porous region is capable of binding or immobilizing a molecule such as a sample molecule or probe. The terms “bind”, “binding”, “immobilize”, “immobilized”, or “affixed”, as used herein are generally interchangeable, and refer to an association between a molecule, including a biomolecule such as a nucleic acid or protein, and a substrate characterized by covalent bonding, intermediate linker molecules, steric hindrance, hybridization or any combination thereof. For example, a molecule can be immobilized to a substrate by covalent bonding directly to a surface of the porous region of the substrate which may or may not be modified to enhance such covalent bonding. Also, the molecule can be immobilized to the substrate by use of a linker molecule between the molecule and the porous region. Molecules can further be immobilized on the substrate by steric hindrance within a substrate. Additionally, molecules can also be immobilized on a substrate through hybridization between an additional molecule, such as a nucleic acid or protein, that is immobilized on the support. Affixing or immobilizing molecules to a substrate can be performed using a covalent linker including, for example, oxidized 3-methyl uridine, an acrylyl group and hexaethylene glycol. Additionally, acrydite oligonucleotide primers may be covalently fixed within a substrate. Various techniques for immobilizing molecules are further discussed below with respect to probe molecules, but are intended to be useful for immobilizing to the substrate any molecule suitable for use in the devices or methods of the present invention.

In another embodiment, a microwell may include a self-assembled monolayer (SAM) comprising a plurality organothiol molecules bonded to a metallic layer. A SAM can be located within the microwell, such as in the porous region or deposited at the microwell bottom. The microwell bottom refers to the region of the microwell where the porous region and the cavity intersect.

SAM are generally depicted as an assembly of organized, closely packed linear molecules. Self-assembled monolayers formed, for example, by the chemisorption of organic molecules on metallic surfaces (e.g., gold) are well characterized synthetic organic monolayers. See, Ulman, An Introductin to Ultrathin Organic Films: From Langmuir-Blodgett to Self-Assembly, Academic Press, San Diego, 1991; Dubois el al., Annu. Rev. Phys. Chem., 43:437 (1992). These monolayers form spontaneously upon contacting an organothiol molecule with a metallic layer as a result of chemisorption of sulfur on the textured surface of the metallic films. The molecules self-organize into a commensurate lattice on the surface of the metallic layer. See, Porter, J. Am. Chem. Soc., 109:3559 (1987); Camillone III, et al., Chem. Phys., 98:3503 (1993); Fenter et al., Science, 266:1216 (1994); 20; Chidsey et al., Langmuir, 6:682 (1990); Sun etal., Thin Solid Films, 242:106 (1994). For monolayers formed from organic molecules, such as an alkylthiol (CH₃(CH₂)ₙSH), the aliphatic chains of the monolayers are extended in the all-trans conformation and typically tilted (e.g., approximately 30 degrees) from the normal of the surface. Because the spacing between sulfur groups on the lattice is, on average, 4.9 angstroms, whereas the van der Waals diameter of an aliphatic chains is only approximately 4 angstroms, the aliphatic chains within these SAMs tilt from the normal so as to come into van der Waals contact and thereby maximize their cohesive dispersive interactions. Studies of the lateral structure within monolayers using X-ray diffraction reveal the existence of domains of size about 100 angstroms, where each domain has one of six different tilt directions relative to the metallic layer. See, for example, Fenter et al., Science, 266:1216 (1994).

Metals that are suitable as coatings comprising a metallic layer of a SAM include, but are not limited to, gold, silver, platinum, palladium, nickel and copper. Silver and gold are preferred, with gold being particularly preferred. The metallic layer can be either continuous or discontinuous. Further, the thickness of the metallic layer can remain constant or can vary over a portion of the microwell. The metal layer is capable of forming a bond or association (e.g., chemisorption, physisorption) with an organic molecule, such as an organosulfer or organothiol (e.g., alkylthiol, sulfide, disulfide). Thus, in one embodiment, a microwell includes a SAM having a plurality of organic molecules attached to a metallic layer. More particularly, a SAM of the invention is a gold metallic layer having a plurality of organothiol molecules attached thereto. In one particular embodiment, a plurality of alkylthiol molecules are attached to the gold metallic layer.

In another embodiment, a microwell of an inventive device or microfabricated substrate can have an electrode positioned in the cavity. The electrode can be operatively connected to a current source, thereby enabling application of a current to the microwell. In particular, an electrode positioned in a cavity is capable of applying an electrical stimulus to the porous region of the microwell with which the electrode is coupled. Such electrode positioning allows various chemical reactions, assays, synthesis reactions, and the like, which may be conducted in the microwell to additionally be influenced electrochemically. Suitable electrode material will be apparent to one skilled in the art and generally is selected from aluminum, gold, silver, tin, copper, platinum, palladium, carbon, and semiconductor materials.

The porous region and the cavity of each microwell are aligned such that the microwell forms a continuous channel extending through the substrate. In this regard, each microwell is capable of forming an “ion bridge” or pathway for the flow of ions between the two sides of the substrate. In one embodiment, an ion bridge is formed where an electrode is positioned in a cavity of a microwell. Additionally, a second electrode can be aligned with the electrode of the cavity and positioned on a side of the substrate opposite the substrate side having a cavity, such as the substrate side having a porous region formed therein. In such an embodiment, the electrodes are separated by a porous region and, when energized, cause ions to flow between the electrodes and through the porous region, thereby creating an ion bridge.

Further, a microwell may optionally include a conductive material deposited in the cavity. A conductive material may be deposited to aid in the formation of an electrochemical gradient in a microwell. Various compositions suitable for use as a conductive material will be apparent to those skilled in the art and include, for example, conductive epoxies (e.g., silver epoxy), electroless nickel plating, conductive gels and polymers, and other conductive materials, some of which are commercially available (see, for example, Epoxy Technology, Billerica Mass.)

In certain embodiments, the device can comprise a micro-electro-mechanical system (MEMS). MEMS are integrated systems including mechanical elements, sensors, actuators, and electronics. All of those components can be manufactured by microfabrication techniques on a common chip, of a silicon-based or equivalent semiconductor substrate (e.g., Voldman et al., Ann. Rev. Biomed. Eng. 1:401-425, 1999). The sensor components of MEMS can be used to measure biological, chemical, optical and/or magnetic phenomena to detect binding signals or fluorescence associated with a microwell of the substrate. The electronics can, where appropriate, process the information from the sensors and control actuator components, such as pumps, valves, heaters, etc. thereby controlling the function of the MEMS.

The electronic components of MEMS can be fabricated using integrated circuit (IC) processes (e.g., CMOS or Bipolar processes). They can be patterned using photolithographic and/or etching methods for computer chip manufacture. The micromechanical components can be fabricated using compatible “micromachining” processes that selectively etch away parts of the substrate or add new structural layers to form the mechanical and/or electromechanical components.

Basic techniques in MEMS manufacture include depositing thin films of material on a substrate, applying a patterned mask on top of the films by some lithographic methods, and selectively etching the films. A thin film can be in the range of a few nanometers to 100 micrometers. Deposition techniques of use can include chemical procedures such as chemical vapor deposition (CVD), electrodeposition, epitaxy and thermal oxidation and physical procedures like physical vapor deposition (PVD) and casting. Methods for manufacture of nanoelectromechanical systems can also be used (See, e.g., Craighead, Science 290:1532-36, 2000.)

In some embodiments, microwells of the device can be connected to various fluid filled compartments, for example microfluidic channels or nanochannels. These and other components of the device can be formed as a single unit, for example in the form of a chip (e.g. semiconductor chips) and/or microcapillary or microfluidic chips. Alternatively, individual components can be separately fabricated and attached together. Any materials known for use in such chips can be used in the disclosed apparatus, for example silicon, silicon dioxide, polydimethyl siloxane (PDMS), polymethylmethacrylate (PMMA), plastic, glass, quartz, etc.

Techniques for batch fabrication of substrates are well known in computer chip manufacture and/or microcapillary chip manufacture. Such substrates can be manufactured by any method known in the art, such as by photolithography and etching, laser ablation, injection molding, casting, molecular beam epitaxy, dip-pen nanolithography, chemical vapor deposition (CVD) fabrication, electron beam or focused ion beam technology or imprinting techniques. Non-limiting examples include conventional molding, dry etching of silicon dioxide; and electron beam lithography. Methods for manufacture of nanoelectromechanical systems can be used for certain embodiments (See, e.g., Craighead, Science 290:1532-36, 2000.). Various forms of microfabricated chips or substrates are commercially available from, e.g., Caliper Technologies Inc. (Mountain View, Calif.) and ACLARA BioSciences Inc. (Mountain View, Calif.).

Following formation of the array of microwells in the substrate, an additional layer, having a structure different from the substrate can optionally be deposited on the substrate at areas initially covered by the mask, such as areas surrounding the microwells. In one embodiment, the additional layer is a “sample containment layer” or a layer that is deposited to prevent the samples or material deposited in the microwells from wicking or spreading to another location on the substrate, such an another microwell. Using this technique, substrates having, on a surface, regions of different chemical characteristics can be produced. Thus, for example, areas on a substrate having an array of adjacent wells can be created with hydrophobicity/hydrophilicity, charge and other chemical characteristics of the deposited sample containment layer. In one embodiment, sample containment layer can be a series of “surrounds”, or distinct deposits confined to areas immediately surrounding individual wells. In another embodiment, a sample containment layer can be a continuous layer that substantially covers the entire area of a substrate surface initially covered by a mask. Techniques for applying an additionally layer will be readily apparent and can include, for example, silk screening, printing, growing, etching, sputtering and the like. Similar substrate configurations are accessible through microprinting a layer with the desired characteristics directly onto the substrate. See, Mrkish, M.; Whitesides, G. M., Ann. Rev. Biophys. Biomol. Struct. 25:55-78 (1996).

A device of the invention may further include a marker which conveys information about the location of the microwells on the substrate. In some embodiments, the markers may be optical markers, such as optical bar codes or fluorescent markers, in another embodiment the markers may be magnetic. In another embodiment, the marker may comprise a multilayered strip comprising a series of alternating reflective and non-reflective surfaces. In such an embodiment, a first layer of the strip is a reflective material, such as aluminum, and a second layer is a protective oxide layer. The marker may be patterned such that a boundary edges of a reflective surface and non-reflective surfaces are aligned a particular microwell, or column or row of microwells on the substrate. For example, a boundary edge may be aligned with a coordinated center line diameter of a column of microwells within the array. Another marker may be printed that is aligned perpendicular to the first encoder mark and at a set distance from the center line diameter of the first microwell in a column. The boundary edge of the second marker can be used as a reference to the center line of the first microwell in the column. Thus, the boundary edges provide fiducial marks on the surface of a substrate to accurately locate a microwell(s). Markers may facilitate use of a device in conjunction with automated equipment, such as to permit alignment of microwells with automated aspirating or dispensing equipment.

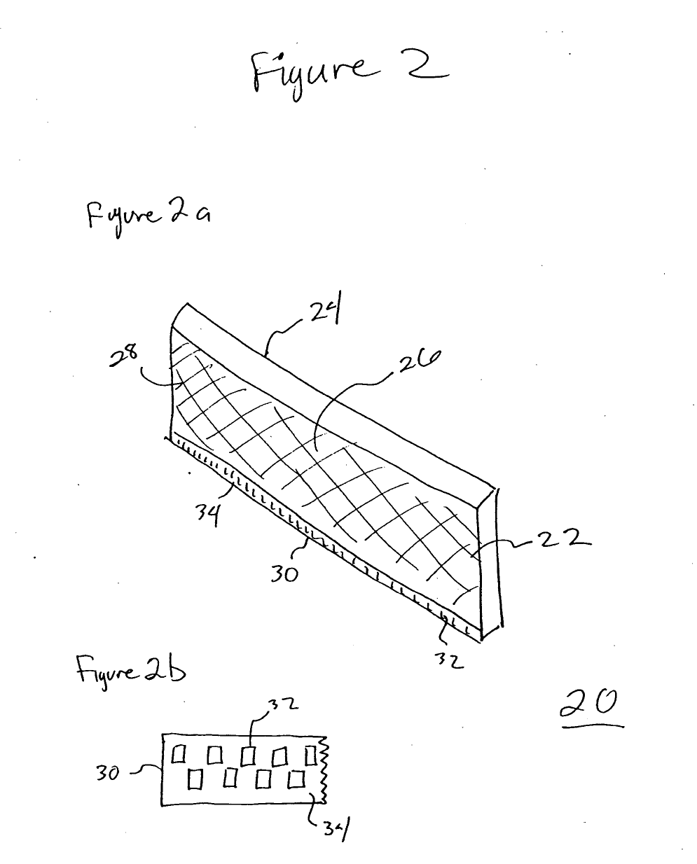

FIGS. 2a and 2b illustrate an embodiment of a device 20 of the invention. The device 20 comprises a first side 22, a second side 24, and an array of microwells 26. The first side 22 includes a sample containment layer 28 and a marker 30. The marker 30 conveys information about the location of the microwells on the substrate, and is capable of directing and coordinating automated equipment addressing the microwells of the device 20. Various configurations and arrangements are available for a marker 30, including for example, optical marks 32 and reflective coating 34, such as an aluminum coating, under the optical marks 32.

Other methods of the invention include methods that are carried out in a device of the invention, or in a substrate having an array of microwells. In one embodiment, such methods include simultaneously conducting a plurality of chemical reactions. These methods include providing a substrate having an array of microwells, each microwell having a porous region formed in a first side of the substrate and capable of binding a sample molecule, where the porous region is a continuous portion of the substrate, extends partially through the substrate, and is formed by selectively removing at least one constituent of the substrate. Each microwell further includes a cavity located at a side of the substrate opposite the first side, with the cavity extending partially through the substrate to intersect the porous region. The methods further include introducing, under suitable reaction conditions, a plurality of test samples into the plurality of microwells of the device, wherein the test samples contain sample molecules as well as necessary reaction components.

Chemical reactions capable of being performed in a device of the invention, including a microfabricated substrate, include, for example, any type of biochemical or molecular biological reaction known to one of skill in the art including, but not limited to, nucleotide sequencing (e.g., chain-termination sequencing, cycle sequencing), amplification reactions (e.g., polymerase chain reactions), transcription, reverse transcription, restriction enzyme digestion, ligation, primer extension, other enzymatic reactions and biological interactions (such as, for example, avidin-biotin, streptavidin-biotin, antibody-antigen and ligand-receptor interactions). In general, any type of reaction involving a biomolecule immobilized to a substrate or solid support, where contacting and/or reacting the immobilized biomolecule with another molecule or biomolecule is desired, can be performed in the device. In addition, multiple micro-volume hybridization reactions can be conducted in the device. In one embodiment, an apparatus is used for very high throughput analysis of chemical samples; for example, in combinatorial chemistry. In another embodiment, the devices disclosed herein will be especially useful in the field of polynucleotide synthesis, for techniques such as the synthesis or production of various length oligonucleotide molecules.

Devices and microfabricated substrates of the present invention also are useful for performing a various range of assays. Such assays are generally based on specific binding reactions are useful for detecting a wide variety of components such as drugs, hormones, enzymes, proteins, antibodies, and infectious agents in various biological fluids and tissue samples. In general, the assays consist of an analyte, a probe for binding the analyte, and a detectable label. Immunological assays, for example, involve reactions between immunoglobulins (antibodies) which are capable of binding with specific antigenic determinants of various compounds and materials (antigens). Other types of reactions include binding between avidin and biotin, protein A and immunoglobulins, lectins and sugar moieties and the like. See, for example, U.S. Pat. No. 4,313,734, issued to Leuvering; U.S. Pat. No. 4,435,504, issued to Zuk; U.S. Pat. Nos. 4,452,901 and 4,960,691, issued to Gordon; and U.S. Pat. No. 3,893,808, issued to Campbell.

These assay techniques provide the ability to detect both the presence and amount of small quantities of analytes and are useful in, for example medical diagnostics and forensic applications.

Thus, one embodiment of the present invention provides a method for detecting an analyte in a test sample or array of test samples. One method includes detecting the presence or amount of an analyte in an array of test samples. The method includes contacting a test sample, under suitable binding conditions, with in the array of samples with a microwell defined in a substrate of the invention. Such a substrate includes an array of microwells, where each microwell includes a porous region and a cavity. The porous region of each microwell is formed in a first side of the substrate by selectively removing at least one substrate constituent. Further, the porous region is a continuous portion of the substrate, and extends partially through the substrate. The cavity of a microwell is located at a side of the substrate opposite the first side, and each cavity extends partially through the substrate to intersect the porous region. Each microwell further includes a probe immobilized to the porous region. The method also includes forming a complex between the probe and the analyte, and detecting the probe-analyte complex, thereby detecting the presence or amount of an analyte in a test sample.

As used herein, the term “probe” refers to molecules which are attached to or immobilized the porous region of a microwell. The probes can interact with the analyte via either attractive or repulsive mechanisms. In one exemplary embodiment, the analyte and the probe form a binding pair, or “complex”, for example, via covalent bonding, ionic bonding, ion pairing, van der Waals association and the like.

Probes can be selected from a wide range of small organic molecules (e.g., drugs, pesticides, toxins, etc.), organic functional groups (e.g., amines, carbonyls, carboxylates, etc.), biomolecules, metals, metal chelates and organometallic compounds.

The above enumerated, and other molecules, can be attached to the porous region of a microwell by methods well-known to those of skill in the art. Ample guidance can be found in literature devoted to, for example, the fields of bioconjugate chemistry and drug delivery.

In other embodiments, the probe is a biomolecule such as a protein, nucleic acid, peptide or an antibody. Biomolecules useful in practicing the present invention can be derived from any source. The biomolecules can be isolated from natural sources or can be produced by synthetic methods. Proteins can be natural proteins or mutated proteins, such as those proteins effected by chemical mutagenesis, site-directed mutagenesis or other means of inducing mutations known to those of skill in the art. Proteins useful in practicing the instant invention include, for example, enzymes, antigens, antibodies and receptors. Antibodies can be either polyclonal or monoclonal, or fragments thereof. Peptides and nucleic acids can be isolated from natural sources or can be wholly or partially synthetic in origin.

In some embodiments, the probe is a polynucleotide. The term “polynucleotide” is used broadly herein to mean a sequence of deoxyribonucleotides or ribonucleotides that are linked together by a phosphodiester bond. For convenience, the term “oligonucleotide” is used herein to refer to a polynucleotide that can be synthesized and/or used as a primer or a probe. Generally, an oligonucleotide useful as a probe or primer that selectively hybridizes to a selected nucleotide sequence is at least about 10 nucleotides in length, usually at least about 15 nucleotides in length, for example between about 15 and about 50 nucleotides in length, up to about 100 nucleotides in length.

A polynucleotide can be RNA or can be DNA, which can be a gene or a portion thereof, a cDNA, a synthetic polydeoxyribonucleic acid sequence, or the like, and can be single stranded or double stranded, as well as a DNA/RNA hybrid. In various embodiments, a polynucleotide, including an oligonucleotide (e.g., a probe or a primer) can contain nucleoside or nucleotide analogs, or a backbone bond other than a phosphodiester bond. In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. However, a polynucleotide or oligonucleotide also can contain nucleotide analogs, including non-naturally occurring synthetic nucleotides or modified naturally occurring nucleotides. Such nucleotide analogs are well known in the art and commercially available, as are polynucleotides containing such nucleotide analogs (Lin et al., Nucl. Acids Res. 22:5220-5234 (1994); Jellinek et al., Biochemistry 34:11363-11372 (1995); Pagratis et al., Nature Biotechnol. 15:68-73 (1997))

As used herein, the term “hybridization” generally refers to “selective hybridization” or “selectively hybridize,” or hybridization under moderately stringent or highly stringent conditions such that a nucleotide sequence preferentially associates with a selected nucleotide sequence over unrelated nucleotide sequences to a large enough extent to be useful in identifying the selected nucleotide sequence. It will be recognized that some amount of non-specific hybridization is unavoidable, but is acceptable provided that hybridization to an analyte nucleotide sequence is sufficiently selective such that it can be distinguished over the non-specific cross-hybridization, for example, at least about 2-fold more selective, generally at least about 3-fold more selective, usually at least about 5-fold more selective, and particularly at least about 10-fold more selective, as determined, for example, by an amount of labeled oligonucleotide that binds to analyte nucleic acid molecule as compared to a nucleic acid molecule other than the analyte molecule, particularly a substantially similar (e.g., homologous) nucleic acid molecule other than the analyte nucleic acid molecule. Conditions that allow for selective hybridization can be determined empirically, or can be estimated based, for example, on the relative GC:AT content of the hybridizing oligonucleotide and the sequence to which it is to hybridize, the length of the hybridizing oligonucleotide, and the number, if any, of mismatches between the oligonucleotide and sequence to which it is to hybridize (see, for example, Sambrook et al., “Molecular Cloning: A laboratory manual (Cold Spring Harbor Laboratory Press 1989), incorporated in its entirety by reference).

An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

Probes which are antibodies can be used to recognize analytes which are proteins, peptides, nucleic acids, saccharides or small molecules such as drugs, herbicides, pesticides, industrial chemicals and agents of war. Methods of raising antibodies for specific molecules are well-known to those of skill in the art. See, U.S. Pat. No. 5,147,786, issued to Feng et al. on Sep. 15, 1992; U.S. Pat. No. 5,334,528, issued to Stanker et al. on Aug. 2, 1994; U.S. Pat. No. 5,686,237, issued to Al-Bayati, M. A. S. on Nov. 11, 1997; and U.S. Pat. No. 5,573,922, issued to Hoess et al. on Nov. 12, 1996. Methods for attaching antibodies to surfaces are also known in the art. See, Delamarche et al. Langmuir, 12:1944-1946 (1996).

By “analyte” is meant any molecule or compound present in a biological sample. An analyte can be in the solid, liquid, gaseous or vapor phase. By “gaseous or vapor phase analyte” is meant a molecule or compound that is present, for example, as a contaminant in a biological sample. It will be recognized that the physical state of the gas or vapor phase can be changed by pressure, temperature as well as by affecting surface tension of a liquid by the presence of or addition of salts etc.

The invention methods may be used to detect the presence of a particular analyte, for example, a nucleic acid, oligonucleotide, protein, enzyme, antibody or antigen. The invention methods may also be used to screen bioactive agents, e.g. drug candidates, for binding to a particular analyte in a biological sample or to detect the presence of agents, such as pollutants, in a biological sample. As discussed above, any analyte for which a probe moiety, such as a peptide, protein, oligonucleotide or aptamer, may be designed can be detected using the invention methods.

The analyte may be a molecule found directly in a sample, such as a bodily fluid from a host. The sample can be examined directly or may be pretreated to render the analyte more readily detectible. Furthermore, the analyte of interest may be determined by detecting an agent probative of the analyte of interest such as a specific binding pair member complementary to the analyte of interest, whose presence will be detected only when the analyte of interest is present in a sample. Thus, the agent probative of the analyte becomes the analyte that is detected in an assay. The bodily fluid can be, for example, urine, blood, plasma, serum, saliva, semen, stool, sputum, cerebral spinal fluid, tears, mucus, and the like.

Another group of exemplary methods uses the invention methods to detect one or more analyte nucleic acid in a sample. Such a method is useful, for example, for detection of infectious agents within a clinical sample, detection of an amplification product derived from genomic DNA or RNA or message RNA, or detection of a gene (cDNA) insert within a clone. For certain methods aimed at detection of an analyte polynucleotide, an oligonucleotide probe is synthesized using methods known in the art. The oligonucleotide probe is then used as a probe to be attached to the biosensor. The oligonucleotide probe is used in a hybridization reaction to allow specific binding of the oligonucleotide probe to an analyte polynucleotide in the sample. The complex formed by binding of the oligonucleotide probe can then be detected using, for example, a detectable label, such as by fluorescence emission or other types of spectroscopy as described herein. Detection of the specific binding of the known oligonucleotide probe to an analyte polynucleotide in the sample provides information regarding the nucleotide sequence of the analyte polynucleotide.

Suitable probes generally include, without limitation, non-polymeric small molecules, antibodies, antigens, polynucleotides, oligonucleotides, receptors, ligands, and the like.

Exemplary non-polymeric small molecules suitable for use as a first probe include, without limitation: avidin, peptido-mimetic compounds, and vancomycin. One class of peptido-mimetic compounds is disclosed in U.S. patent application Ser. No. 09/568,403 to Miller et al., filed May 10, 2000. A peptido-mimetic compound that binds to lipopolysaccharide is a tetratryptophan ter-cyclopentane. Other peptidomimetic compounds can also be employed.

Exemplary polypeptides suitable for use as a first probe include, without limitation, a receptor for a cell surface molecule or fragment thereof, an antibody or fragment thereof, peptide monobodies of the type disclosed in U.S. patent application Ser. No. 09/096,749 to Koide, filed Jun. 12, 1998, and U.S. patent application Ser. No. 10/006,760 to Koide, filed Nov. 19, 2001; a lipopolysacchardide-binding polypeptide; a peptidoglycan-binding polypeptide; a carbohydrate-binding polypeptide; a phosphate-binding polypeptide; a nucleic acid-binding polypeptide; and polypeptides that specifically bind to a protein-containing analyte. In one embodiment, the first probes are antibodies specific for a particular protein-containing analyte or a particular class or family of protein-containing analytes.

Exemplary oligonucleotide first probes can be DNA, RNA, or modified (e.g., propynylated) oligonucleotides of the type disclosed in Barnes et al., J. Am. Chem. Soc. 123:4107-4118 (2001), and Barnes et al., J. Am. Chem. Soc. 123:9186-9187 (2001). The oligonucleotide probes can be any length that is suitable to provide specificity for the intended analyte. Typically, oligonucleotide probes that do not contain modified nucleotides will be at least about 12 to about 100 nucleotides in length. For oligonucleotides that contain modified bases, a length of at least 7 nucleotides, up to about 100 nucleotides is suitable.

Analyte molecules that can be bound by probes include, without limitation: proteins (including without limitation enzymes, antibodies or fragments thereof), glycoproteins, peptidoglycans, carbohydrates, lipoproteins, a lipoteichoic acid, lipid A, phosphates, nucleic acids that are expressed by certain pathogens (e.g., bacteria, viruses, multicellular fungi, yeasts, protozoans, multicellular parasites, etc.), or organic compounds such as naturally occurring toxins or organic warfare agents, etc. These analyte molecules can be detected from any source, including bodily fluids, food samples, water samples, homogenized tissue from organisms, etc.

A number of strategies are available for attaching the one or more first probes to the porous region, depending upon the type of probe that is ultimately to be attached thereto. Because of the porosity of this region of a microwell of the structure, the probes can be bound to the exposed surfaces throughout the porous region of a microwell.

The available strategies for attaching the one or more first probes include, without limitation, covalently bonding a probe to the surface of the semiconductor structure, ionically associating the probe with the surface of the semiconductor structure, adsorbing the probe onto the porous region, or the like. Such association can also include covalently or noncovalently attaching the probe to another moiety (of a coupling agent), which in turn is covalently or non-covalently attached to the surface of the semiconductor structure.

Basically, the oxidized and hydrolyzed surface of the substrate is first functionalized (e.g., primed) with a coupling agent which is attached to the surface thereof. This is achieved by providing a coupling agent precursor and then covalently or non-covalently binding the coupling agent precursor to the porous region. Once the porous region has been functionalized, the probe is exposed to the functional group attached to the porous region under conditions effective to (i) covalently or non-covalently bind to the coupling agent or (ii) displace the coupling agent such that the probe covalently or non-covalently binds directly to the porous. The binding of the first probe to the semiconductor structure is carried out under conditions that are effective to allow the one or more analyte-binding groups thereon to remain available for binding to the analyte molecule.

Suitable coupling agent precursors include, without limitation, silanes functionalized with an epoxide group, a thiol, or an alkenyl; and halide containing compounds.

Silanes include a first moiety which binds to the surface of the semiconductor structure and a second moiety which binds to the probe. Preferred silanes include, without limitation, 3-glycidoxypropyltrialkoxy-silanes with C1-6 alkoxy groups, trialkoxy(oxiranylalkyl)silanes with C2-12 alkyl groups and C1-6 alkoxy groups, 2-(1,2-epoxycyclohexyl)ethyltr-ialkoxysilane with C1-6 alkoxy groups, 3-butenyl trialkoxysilanes with C1-6 alkoxy groups, alkenyltrialkoxysilanes with C2-12 alkenyl groups and C1-6 alkoxy groups, tris[(1-methylethenyl)oxy]3-oxiranylalkyl silanes with C2-12 alkyl groups, [5-(3,3-dimethyloxiranyl)-3-methyl-2-pentenyl]trialkoxysilane with C1-6 alkoxy groups, (2,3-oxiranediyldi-2,1-ethanediyl)b-is-triethoxysilane, trialkoxy[2-(3-methyloxiranyl)alkyl]silane with C1-6 alkoxy groups and C2-12 alkyl groups, trimethoxy[2-[3-(17,17,17-trifluoro-heptadecyl)oxiranyl]ethyl]silane, tributoxy[3-[3-(chloromethyl)oxiranyl]-2-methylpropyl]silane, and combinations thereof. Silanes can be coupled to the semiconductor structure according to a silanization reaction scheme for which the conditions are well known to those of skill in the art.

Halides can also be coupled to the semiconductor structure under conditions well known to those of skill in the art.

The detectable label is often necessary because the results of specific binding reactions, or formation of a probe-analyte complex, are frequently not directly observable. A variety of detectable labels have been devised for determining the presence of a reaction. Detectable labels have involved well known techniques including radiolabeling and the use of chromophores, fluorophores and enzyme labels. Radiolabels can be detected by radiation detectors. Chromophores and fluorophores have been detected by use of spectrophotometers or the naked eye. Redox active groups can be detected by electroanalytical methods. Biotin can be detected by its well-know binding to avidin or strepavidin. The avidin or strepavidin can itself be labeled with any of the labels described herein. Where members of a specific binding pair or complex are tagged with an enzyme label, their presence may be detected by the enzymatic activation of a reaction system wherein a compound such as a dyestuff, is activated to produce a detectable signal.

Thus, in particular embodiments, the label includes fluorescent groups, chromophoric groups, radioactive groups, redox active groups, biotin, enzyme labels and combinations thereof.

The labels in the present invention can be primary labels (where the label comprises an element which is detected directly) or secondary labels (where the detected label binds to a primary label, e.g., as is common in immunological labeling). An introduction to labels, labeling procedures and detection of labels is found in Polak et al., Introduction to Immunocytochemistry, 2nd Ed., Springer Verlag, N.Y., (1977), and in Haugland, Handbook of Fluorescent Probes and Research Chemicals, a combined handbook and catalogue Published by Molecular Probes, Inc., Eugene, Oreg.(1996).

Primary and secondary labels can include undetected elements as well as detected elements. Useful primary and secondary labels in the present invention can include spectral labels such as fluorescent dyes (e.g., fluorescein and derivatives such as fluorescein isothiocyanate (FITC) and Oregon Green.TM., rhodamine and derivatives (e.g., Texas red, tetrarhodimine isothiocynate (TRITC), etc.), dixogenin, biotin, phycoerythrin, AMCA, CyDyes.TM., and the like), radiolabels (e.g., . ³H, ¹²⁵I, ³⁵S, ¹⁴C, ³²P, etc.), enzymes (e.g., horse-radish peroxidase, alkaline phosphatase etc.) spectral colorimetric labels such as colloidal gold or colored glass or plastic (e.g. polystyrene, polypropylene, latex, etc.) beads. The label can be coupled directly or indirectly to a component of the detection assay (e.g., a nucleic acid) according to methods well known in the art. As indicated above, a wide variety of labels can be used, with the choice of label depending on sensitivity required, ease of conjugation with the compound, stability requirements, available instrumentation, and disposal provisions.

In general, a detector which monitors formation of a probe-analyte complex is adapted to the particular label which is used. Typical detectors include spectrophotometers, phototubes and photodiodes, potentiostats, microscopes, scintillation counters, cameras, film and the like, as well as combinations thereof. Examples of suitable detectors are widely available from a variety of commercial sources known to persons of skill. Commonly, an optical image of an analyte comprising bound label is digitized for subsequent computer analysis.

Most typically, the amount of analyte present is measured by quantitating the amount of label fixed to the material of the invention following a binding event. Means of detecting and quantitating labels are well known to those of skill in the art. Thus, for example, where the label is a radioactive label, means for detection include a scintillation counter or photographic film as in autoradiography. Where the label is optically detectable, typical detectors include microscopes, cameras, phototubes and photodiodes. Many other detection systems are widely available.

Immunological assays generally include, for example, competitive binding assays, where labeled reagents and unlabeled analyte compounds compete for binding sites on a binding material. After an incubation period, unbound materials are washed off and the amount of labeled reagent bound to the site is compared to reference amounts for determination of the analyte concentration in the sample solution. Another type of immunological assay is known as a sandwich assay and generally involves contacting an analyte sample solution to a microwell comprising a first probe immunologically specific for that analyte. A second solution comprising an second labeled probe of the same type (antigen or antibody) as the first probe is then added to the assay. The second probe will bind to any analyte which is bound to the first probe. The assay system is then subjected to a wash step to remove second probe which failed to bind with the analyte and the amount of second probe remaining is ordinarily proportional to the amount of bound analyte.

An exemplary immunoassay method using the microfabricated substrate of the invention employs the porous regions of a microwell, with an attached probe adsorbed or bound to the porous surface of the porous region either through covalent or noncovalent attachment.

The microfabricated substrate of the invention can also be used as a solid support for a variety of syntheses reactions. The substrates are useful supports for synthesis of small organic molecules, polymers, nucleic acids, peptides and the like. See, for example, Kaldor et al., “Synthetic Organic Chemistry on Solid Support” In, Combinatorial Chemistry and Molecular Diversity in Drug Discovery, Gordon et al., Eds., Wiley-Liss, N.Y., 1998.

Thus, in another embodiment of the invention provides methods of assembling a compound or a plurality of compounds. The method includes providing a substrate having an array of microwells, each microwell having a porous region formed in a first side of the substrate and capable of binding a component of a compound, where the porous region is a continuous portion of the substrate, extends partially through the substrate, and is formed by selectively removing at least one constituent of the substrate. Each microwell further includes a cavity located at a side of the substrate opposite the first side, with the cavity extending partially through the substrate to intersect the porous region. The method further includes adding a first component of the compound into a plurality of microwells of the substrate, such that the added first component is immobilized to porous regions of the microwells; adding a second component of the compound to the microwells; and reacting, within the plurality of microwells, the first component and the second component to form a product, thereby assembling a plurality of compounds.

In this aspect, the microfabricated substrate is used as a solid phase synthesis support. Use of a solid support for synthesis reactions, in general, are well known in the art. For example, solid supports are widely used to prepare, peptides, nucleic acids, oligosaccharides and small organic molecules, for example. See, Hernkens et al., Tetrahedron, 52:4527-4554 (1996); Leznoff et al., Acc. Chem. Res., 11:327-333 (1978); Frechet et al., J. Am. Chem. Soc., 86:5163-5165 (1971); Kick et al., J. Med Chem., 38:1427-1430 (1995); Atherton et al., Solid-Phase Peptide Synthesis: A Practical Approach, IRL, Oxford, UK, 1989.

In another embodiment for performing synthesis reactions, a device including a substrate having an array of microwells is used a miniature oligonucleotide synthesizer, utilizing standard phosphoramidite chemistry. Phosphophoramidite chemistry consists of repeating a synthesis protocol of four basic steps: deblock, couple, cap, and oxidize. See, Gait, M. J. (1984). Oligonucleotide synthesis: A practical approach. Chapters 1 and 4, Oxford University Press, New York, N.Y., which is hereby incorporated by reference. Using such techniques, a different oligonucleotide can be synthesized at each hole in a multi-step process. The array is exposed to a nucleotide monomer and the appropriate reagents in a step-wise manner, thereby synthesizing in each well an oligonucleotide of the desired length.

In this aspect, one compound can be prepared using one or a plurality of particles. Alternatively, an array of compounds can be synthesized and, preferable screened utilizing the particles of the invention. Further, the addition of components can be repeated using the same or different components as necessary to assemble the desired compound.

The invention provides kits and integrated systems for practicing the various aspects and embodiments of the present invention, including producing the microfabricated substrates and devices utilizing such, performing chemical reactions, performing the syntheses and practicing the assays described herein.

The invention provides kits for practicing the methods noted above. The kits can include any of the devices and/or microfabricated substrates noted above, and optionally further include additional components such as instructions to practice the methods, one or more containers or compartments (e.g., to hold the particulate material, nucleic acids, antibodies, inhibitors or the like), an automated equipment or robotic armature for dispensing, mixing, etc., kit components, or the like.

Therefore, in one aspect of the invention, a kit comprising a device for performing chemical reactions is provided. The device of the kit includes a substrate having an array of microwells. Each microwell of the array includes a porous region and a cavity. The porous region is formed in a first side of the substrate and capable of binding sample molecules, wherein the porous region is a continuous portion of the substrate, extends partially through the substrate, and is formed by selectively removing at least one constituent of the substrate. The cavity of each microwell is located at a side of the substrate opposite the first side and extending partially through the substrate to intersect the porous region. The kit further includes a reaction component packaged in a suitable container. The reaction component can be a reagent for performing various reactions, including ligation reactions, primer extension reactions, nucleotide sequencing reactions, restriction endonuclease digestions, oligonucleotide syntheses, hybridization reactions and biomolecular interactions.

The invention also provides integrated systems for performing the methods disclosed herein. For example, in the assembly of devices, microfabrication of substrates, or performing chemical reactions, syntheses, or assays, the delivery of individual compounds or compound components is accomplished by means of an automated equipment which transfers fluid from a source to a destination, a controller which controls the automated equipment, a label detector, a data storage unit which records label detection, and an assay component such as a microfabricated substrate having an array of microwells. When a labeled compound is used, it is detected by means of the label detector.

Optical images viewed (and, optionally, recorded) by a camera or other recording device (e.g., a photodiode and data storage device) are optionally further processed in any of the embodiments herein, e.g., by digitizing the image and storing and analyzing the image on a computer. A variety of commercially available peripheral equipment and software is available for digitizing, storing and analyzing a digitized video or digitized optical image, e.g., using PC, MACINTOSH™, or other computers, such as UNIX based computers.

The following examples are intended to illustrate but not limit the invention.

### EXAMPLE 1

### Production of a Substrate having an Array of Microwells

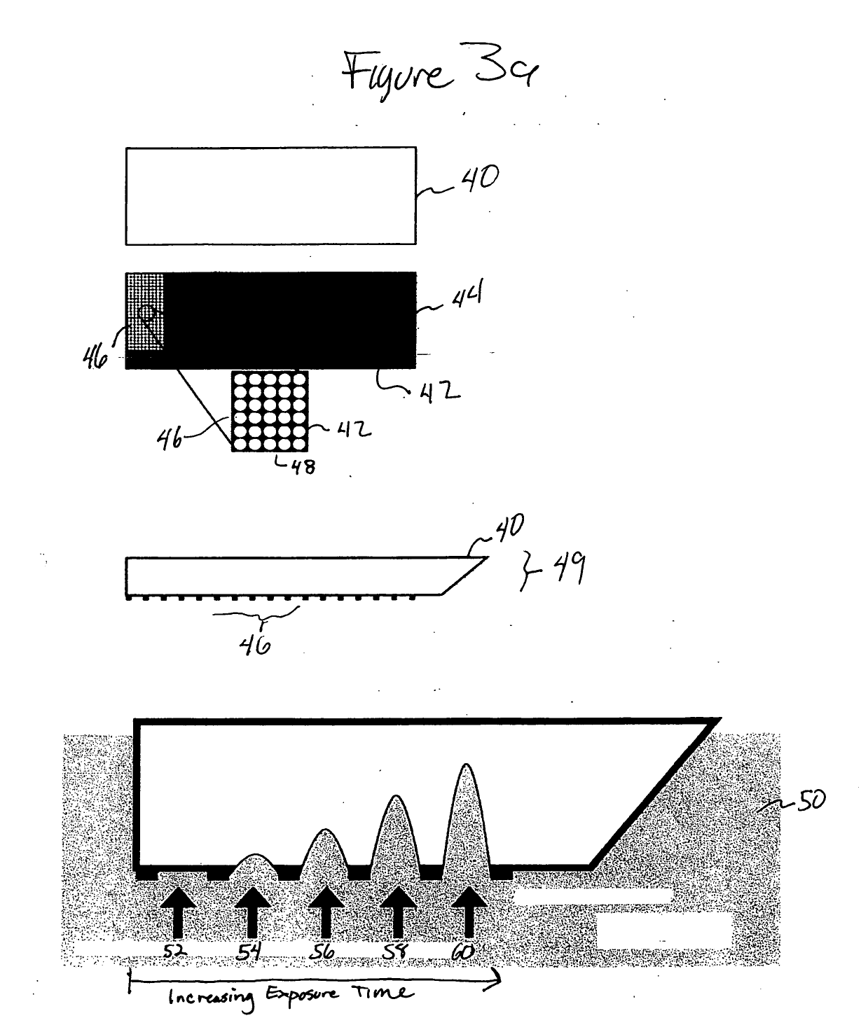

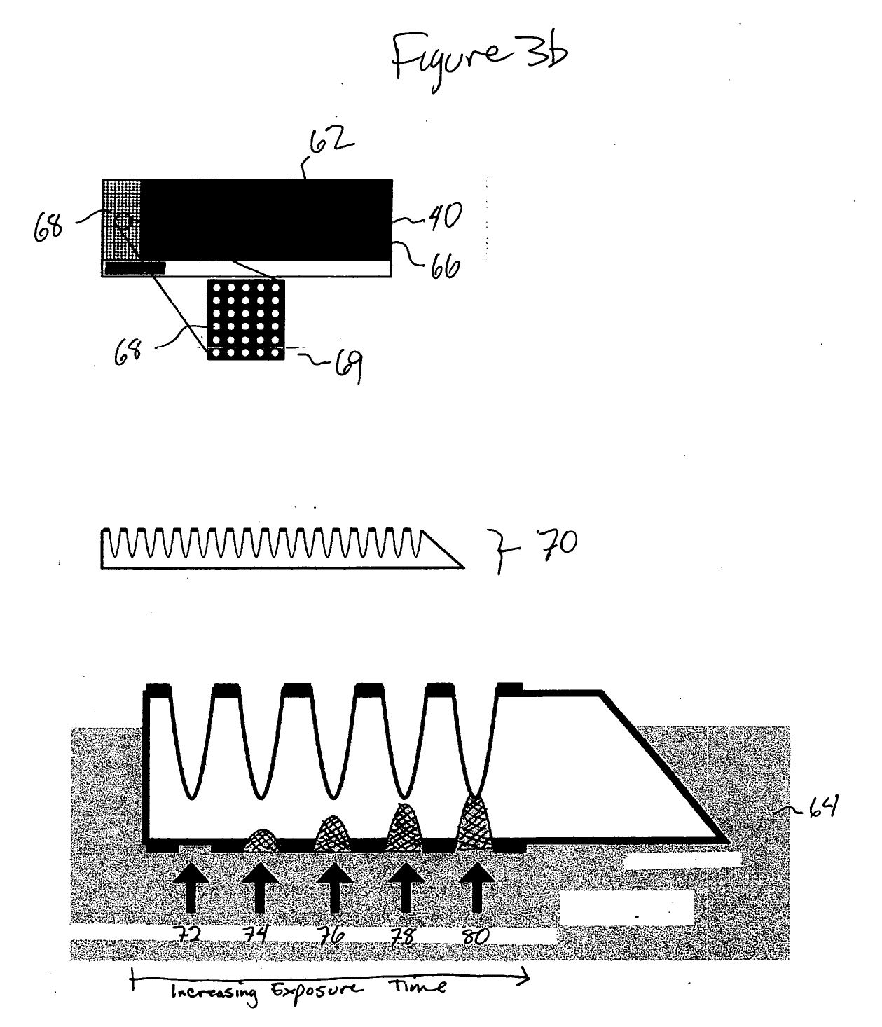

FIGS. 3a and 3b illustrate one method of producing a substrate having an array of microwells according to an embodiment of the invention. In the first step, a suitable substrate 40 is provided. Second, a mask is applied to the a side of the substrate. A mask 42 is applied to a second side 44 of the substrate 40. The mask 42 contains a plurality of openings 46, further illustrated in a expanded window 48, which selectively allows exposure of the second side 44 of the substrate 40 to an etchant. A side view 49 of the substrate illustrating the openings of the mask is shown. Next, the substrate 40 is at least partially immersed in an etching bath. The etchant contacts the substrate areas not covered by the mask, thereby removing substrate material and forming cavities at openings 52, 54, 56, 58, 60 in selected areas. FIG. 3a shows exposure for increasing amounts of time and illustrates that the degree to which the substrate material is etched is at least partially dependent on the amount of time the substrate is exposed to the etchant. The etching step is complete when the cavities reach a desired depth.

Following the etching step, a masking layer is applied to the first side 62 of the substrate 40 and the substrate is at least partially immersed in a leaching bath 64. Here, the second side 44 is opposite the first side 62. The masking layer 66 on the first side 62 comprises openings 68, further illustrated in an expanded window 69, which allow selective exposure of the first side 62 of the substrate material to leaching chemicals. The openings 68 defined by the mask 66 on the first side 62 are typically aligned with the openings 46 defined by the mask 42 on the second side 44 of the substrate 40. In one embodiment of the invention, the mask 42 applied to the second side 44 will define larger openings 46 than the openings 68 defined by the mask 66 applied to the first side 62. A side view 70 of the etched substrate 40, illustrating the plurality of cavities, is shown. The leaching bath 64 selectively removes constituents of the substrate 40, thereby forming porous regions at each opening 72, 74, 76, 78, 80 of the mask 66. FIG. 3b further illustrates, by showing increasing amounts of exposure times, that the degree to which a porous region extends through the substrate 40 is at least partially dependent on the amount of time the substrate is exposed to the leachant. The leaching step is complete when the porous region reaches the corresponding cavities on the second side 44 of the substrate 40.

Although the invention has been described with reference to the above example, it will be understood that modifications and variations are encompassed within the spirit and scope of the invention. Accordingly, the invention is limited only by the following claims.

1. A device for performing chemical reactions, comprising a substrate having a first side, a second side and an array of microwells, each microwell comprising a porous region:
(a) defined by a continuous portion of the substrate;
(b) capable of binding sample molecules;
(b) defined in the first side of the substrate;
(c) formed by selective removal of a substrate constituent; and
(d) extending partially through the substrate.

2. The device of claim 1, wherein each microwell holds a sample such that a liquid sample in one microwell does not intermix with a liquid sample from another microwell.

3. The device of claim 1, wherein pores within the porous region are at least 2.5 nanometers in size.

4. The device of claim 1, wherein pores within the porous region are between 7.5 and 60.0 nanometers in size.

5. The device of claim 1, wherein the substrate is a borosilicate glass.

6. The device of claim 5, wherein the porous region is formed by heating the substrate, thereby causing ion constituent of the substrate to coalesce, and removing coalesced constituent by chemical dissolution.

7. The device of claim 1, further comprising a cavity located on the second side of the substrate and extending partially through the substrate to intersect the porous region.

8. The device of claim 7, wherein the porous region and the cavity are aligned such that the microwell forms a continuous channel extending through the substrate.

9. The device of claim 7, wherein at least a potion of the microwell further comprises a reactive monolayer deposited thereon.

10. The device of claim 9, wherein the reactive monolayer comprises a plurality of organothiol molecules covalently bonded to a metallic layer.

11. The device of claim 10, wherein the organothiol molecules are alkylthiols.

12. The device of claim 10, wherein the metallic layer comprises gold.

13. The device of claim 7, further comprising an electrode coupled with at least one microwell.

14. The device of claim 13, wherein the electrode is positioned in the cavity.

15. The device of claim 14, wherein the electrode is capable of applying an electrical stimulus to the porous region of the microwell with which the electrode is coupled.

16. The device of claim 15, wherein electrode comprises a material selected from the group consisting of aluminum, gold, silver, tin, copper, platinum, palladium, carbon, and semiconductor materials.

17. The device of claim 8, wherein each microwell is capable of forming an ion bridge between the two sides of the substrate.

18. The device of claim 17, further comprising a conductive material deposited in the cavity.

19. The device of claim 18, wherein the conductive material comprises a conductive epoxy, electroless nickel plating, conductive gel, or conductive polymer.

20. The device of claim 1, further comprising a sample containment layer deposited on the first side such that a sample present in one microwell does not intermix with a sample present in another microwell.

21. The device of claim 20, wherein the sample containment layer is hydrophobic.

22. The device of claim 20, wherin the sample containment layer is hydrophilic.

23. The device of claim 1, further comprising a marker that conveys information about the location of the microwells on the substrate.

24. The device of claim 23, wherein the marker is a bar code.

25. The device of claim 23, wherein the marker comprises a series of alternating reflective and non-reflective surfaces.

26. The device of claim 1, further comprising a means for conveying information about the location of the microwells on the substrate.

27. A device for performing chemical reactions comprising a substrate having an array of microwells, each microwell comprising:
(a) a porous region, formed in a first side of the substrate capable of binding sample molecules, wherein the porous region is a continuous portion of the substrate, extends partially through the substrate, and is formed by selectively removing at least one constituent of the substrate; and
(b) a cavity located at a side of the substrate opposite the first side and extending partially through the substrate to intersect the porous region.

28. The device of claim 27, wherein each microwell holds a sample such that a liquid sample in one microwell does not intermix with a liquid sample from another microwell.

29. The device of claim 27, wherein pores within the porous region are at least 2.5 nanometers in size.

30. The device of claim 27, wherein pores within the porous region are between 7.5 and 60.0 nanometers in size.

31. The device of claim 27, wherein the substrate is a borosilicate glass.

32. The device of claim 31, wherein the porous region is formed by heating the substrate, thereby causing ion constituent of the substrate to coalesce, and removing coalesced constituent by chemical dissolution.

33. The device of claim 27, wherein the porous region and the cavity are aligned such that the microwell forms a continuous channel extending through the substrate.

34. The device of claim 27, wherein at least a potion of the microwell further comprises a reactive monolayer deposited thereon.

35. The device of claim 34, wherein the reactive monolayer comprises a plurality of organothiol molecules covalently bonded to a metallic layer.

36. The device of claim 35, wherein the organothiol molecules are alkylthiols.

37. The device of claim 35, wherein the metallic layer comprises gold.

38. The device of claim 27, further comprising an electrode coupled with at least one microwell.

39. The device of claim 38, wherein the electrode is positioned in the cavity.

40. The device of claim 39, wherein the electrode is capable of applying an electrical stimulus to the porous region of each microwell with which the electrode is coupled.

41. The device of claim 40, wherein electrode comprises a material selected from the group consisting of aluminum, gold, silver, tin, copper, platinum, palladium, carbon, and semiconductor materials.

42. The device of claim 33, wherein the microwell capable of forming an ion bridge between two sides of the substrate.

43. The device of claim 42, further comprising a conductive material deposited in the cavity.

44. The device of claim 43, wherein the conductive material comprises a conductive epoxy, electroless nickel plating, conductive gel, or conductive polymer.

45. The device of claim 27, further comprising a sample containment layer deposited on the first side such that a sample present in one microwell does not intermix with a sample present in another microwell.

46. The device of claim 45, wherein the sample containment layer is hydrophobic.

47. The device of claim 45, wherin the sample containment layer is hydrophilic.

48. The device of claim 27, further comprising a marker that conveys information about the location of the microwells on the substrate.

49. The device of claim 48, wherein the marker is a bar code.

50. The device of claim 48, wherein the marker comprises a series of alternating reflective and non-reflective surfaces.

51. The device of claim 27, further comprising at least one component of a chemical reaction to be carried out in the device.

52. The device of claim 51, wherein component is immobilized to the porous region.

53. The device of claim 51, wherein the component is a reagent used in a oligonucleotide synthesis reaction.

54. The device of claim 53, wherein the reagent is a nucleic acid.

55. A method of producing a device for performing an array of chemical reactions, the method comprising: providing a substrate having a first side and a second side, and forming an array of microwells in the substrate, wherein the microwells are formed by:
(a) selectively leaching defined areas on the first side of the substrate, thereby forming a plurality of porous regions that are a continuous portion of the substrate and extend partially through the substrate; and
(b) selectively etching defined areas of the second side of the substrate, thereby forming a plurality of cavities, wherein each cavity extends partially through the substrate to intersect with a porous region.

56. The method of 55, wherein the substrate is borosilicate glass.

57. The method of 55, wherein selectively leaching includes the steps of applying a mask to the first side of the substrate and contacting the first side with a leachant.

58. The method of 55, wherein selectively etching includes the steps of applying a mask to the second side of the substrate and contacting the second side with an etchant.

59. The method of 55, further comprising contacting the porous region with an etchant, thereby increasing pore size in the porous region.

60. The method of 55, further comprising immobilizing a sample molecule in the porous region.

61. A method of simultaneously conducting a plurality of chemical reactions, the method comprising:
(a) providing a substrate having an array of microwells, each microwell comprising:
(1) a porous region, formed in a first side of the substrate capable of binding sample molecules, wherein the porous region is a continuous portion of the substrate, extends partially through the substrate, and is formed by selectively removing at least one constituent of the substrate;
(2) a cavity located at a side of the substrate opposite the first side and extending partially through the substrate to intersect the porous region;
(b) introducing, under suitable reaction conditions, a plurality of test samples into a plurality of microwells of the substrate, wherein the test samples contain necessary reaction components, thereby conducting a plurality of chemical reactions.

62. The method of 61, wherein the chemical reactions are selected from the group consisting of ligation reactions, primer extension reactions, nucleotide sequencing reactions, restriction endonuclease digestions, biological interactions, oligonucleotide synthesis reactions, and polynucleotide hybridization reactions.

63. The method of 62, wherein the biological interactions are avidin-biotin interactions, antigen-antibody interactions, enzyme-substrate reactions, ligand-receptor interactions.

64. The method of 61, wherein the chemical reaction is a phosphoamidite chemical reaction for oligonucleotide synthesis.

65. The method of 61, wherein the test samples are immobilized to the porous region of a microwell.

66. A method of detecting an analyte in a plurality of test samples, the method comprising:
(a) contacting each test sample in a plurality of test samples with a microwell defined a substrate having an array of microwells, each microwell comprising:
(1) a porous region, formed in a first side of the substrate capable of binding sample molecules, wherein the porous region is a continuous portion of the substrate, extends partially through the substrate, and is formed by selectively removing at least one constituent of the substrate;
(2) a cavity located at a side of the substrate opposite the first side and extending partially through the substrate to intersect the porous region; and
(3) a probe immobilized to the porous region;
(b) forming a complex between the probe and the analyte; and
(c) detecting, in each microwell contacted with a test sample, the probe-analyte complex, thereby detecting the analyte in a plurality of test samples.

67. The method of claim 66, wherein the test sample is a bodily fluid, a suspension of solids in an aqueous solution, a cell extract, or a tissue homogenate.

68. The method of claim 67, wherein the bodily fluid is selected from urine, blood, plasma, serum, saliva, semen, stool, sputum, cerebral spinal fluid, tears, or mucus.

69. The method of claim 66, wherein the probe is selected from small molecules, organic functional groups, biomolecules, metals, metal chelates, and organometallic compounds.

70. The method of claim 69, wherein the probe is a biomolecule selected from a protein polynucleotide, peptide, antibody, or fragment thereof.

71. A method of assembling a plurality of compounds, comprising:
(a) providing a substrate having an array of microwells, each microwell comprising:
(1) a porous region, formed in a first side of the substrate capable of binding sample molecules, wherein the porous region is a continuous portion of the substrate, extends partially through the substrate, and is formed by selectively removing at least one constituent of the substrate;
(2) a cavity located at a side of the substrate opposite the first side and extending partially through the substrate to intersect the porous region;
(b) adding a first component of the compound to a plurality of microwells, such that the first component binds to each porous region of the microwells;
(c) adding a second component of the compound to the microwells; and
(d) reacting the first component and second component to form a product in each of the plurality of microwells, thereby assembling a plurality of compounds.

72. The claim of 71, further comprising the steps of adding an additional component of the compound to the microwells and reacting the additional component of the compound with the product.

73. The claim of 72, further comprising repeating the steps of G2 to produce a compound of the desired length.

74. The method of claim 71, wherein the compound is a peptide or a oligonucleotide.

75. The method of claim 71, wherein the components of the compound are amino acids or nucleic acids.

76. The method of claim 71, wherein the product is a polypeptide or oligonucleotide.

77. A kit comprising a device for performing chemical reactions, the device comprising a substrate having an array of microwells, each microwell having:
(a) a porous region formed in a first side of the substrate and capable of binding sample molecules, wherein the porous region is a continuous portion of the substrate, extends partially through the substrate, and is formed by selectively removing at least one constituent of the substrate; and
(b) a cavity located at a side of the substrate opposite the first side and extending partially through the substrate to intersect the porous region; and further comprising a reaction component packaged in a suitable container.

78. The kit of claim 77, wherein the reaction component is a reagent for performing a reaction selected from the group consisting of ligation reactions, primer extension reactions, nucleotide sequencing reactions, restriction endonuclease digestions, oligonucleotide synthesis, hybridization reactions and biomolecular interactions.

79. The device of claim 77, wherein the substrate is a borosilicate glass.

80. The device of claim 77, further comprising an electrode coupled with at least one microwell and capable of applying an electrical stimulus to the porous region of the microwell with which the electrode is coupled.

81. The device of claim 77, further comprising a sample containment layer deposited on the first side such that a sample present in one microwell does not intermix with a sample present in another microwell.

82. The device of claim 77, further comprising a marker conveying information about the location of the microwells on the substrate.
